(19) United States
(12) Patent Application Publication
(10) Pub. No.: US 2018/0206860 A1
(43) Pub. Date: Jul. 26, 2018

(54) DEVICES AND METHODS FOR INTRA-OPERATIVE SPINAL ALIGNMENT

(71) Applicant: OrthAlign, Inc., Aliso Viejo, CA (US)

(72) Inventors: Nicholas van der Walt, Laguna Hills, CA (US); Jonathan Nielsen, Aliso Viejo, CA (US)

(21) Appl. No.: 15/674,802

(22) Filed: Aug. 11, 2017

Related U.S. Application Data
(63) Continuation of application No. 14/399,046, filed on Nov. 5, 2014, now abandoned.
(60) Provisional application No. 61/643,979, filed on May 8, 2012.

(51) Int. Cl. A61B 17/17; A61B 34/20; A61B 17/70; A61B 34/00; A61B 90/00; A61B 17/88

(52) U.S. Cl. CPC A61B 17/1757; A61B 34/20; A61B 17/7082; A61B 17/7077; A61B 2090/363; A61B 2034/2048; A61B 2090/067; A61B 2090/372; A61B 17/8866; A61B 34/25

(57) ABSTRACT

The present invention provides a spinal alignment system comprising at least one vertebral coupler, at least one reference device and a surgical orientation device. The present invention further provides methods using the spinal alignment system to assist in alignment of the spine during a surgical procedure to correct a deformity due to trauma or degeneration. The present invention also provides a pedicle screw navigation system comprising of a pedicle screw navigator, a first reference device, a second reference device and a surgical orientation device. The present invention provides methods using the pedicle screw navigation system to assist in determining the proper orientation of a pedicle screw during spinal surgery.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The present application includes inventions that provide devices and/or methods for intraoperatively measuring intervertebral alignment and determining the proper orientation for an implant such as a pedicle screw.

### Description of the Related Art

The human spinal column is a chain of discrete bones allowing substantial flexibility and motion, while protecting nervous and vascular structures running within and around the spinal column. This column extends from the cervical (neck) region, through the thoracic and lumbar regions, to the sacrum at the base of the spine.

Motion between adjacent bones (vertebrae) is accommodated by two articulating cartilage-on-cartilage facet joints posterior to the vertebral body, and by compliant discs between adjacent vertebral bodies. In addition to these structures, each vertebra includes bony protuberances to which tendons and ligaments attach to the vertebrae. Pedicles connect the vertebral bodies to the posterior structures.

Normal spinal alignment is often disrupted as a result of trauma or disease. Degenerative deformity and vertebral fractures usually require surgical intervention and fixation of the spine. Typically, at least two adjacent vertebrae are fused to each other by means of instrumentation affixed to the vertebrae with screws, clamps, or hooks, and/or by inter-vertebral cages that encourage skeletal fusion of the adjacent bones.

Posterior fixation is most often accomplished via stiff rods spanning the affected vertebrae, and fixed to the vertebrae with screws entering through the strong bone of the pedicles. Any screws placed in the vertebrae must be carefully positioned and aligned to avoid injuring the adjacent nervous and vascular structures. Various mechanical or electronic (e.g. StealthStation™ Treatment Guidance System available from Medtronic, Inc.) guidance systems have been developed to aid the surgeon in accurately placing screws in the vertebral bone.

The fixation instrumentation applied to the spine must be adjusted intra-operatively to achieve optimal spinal alignment. Restoration of sagittal, coronal, or transverse plane alignment in these cases is based on approximate correction targets for each spinal level, which are derived from pre-operative radiographs. These correction targets are, in turn, derived from generally-accepted ideal global alignments of the spine in the sagittal, transverse, and coronal planes. Verification of alignment is typically not performed until post-operative radiographs are available so alignment mistakes may not be discovered until after the operation. Accordingly, there is a need for devices and methods to quantitatively measure the relative alignment of adjacent vertebrae and/or the global alignment of longer segments of the spine during the operation so verification of alignment(s) can be performed or intra-operatively thereby avoiding alignment mistakes.

### SUMMARY OF THE INVENTION

The present invention provides, in certain embodiments, a spinal alignment system comprising of at least one vertebral coupler, at least one reference device and a surgical orientation device. The present invention further provides methods to use the spinal alignment system of the present invention to assist in alignment of the spine during a surgical procedure to correct a deformity due to trauma or degeneration.

The present invention provides, in certain embodiments, a pedicle screw navigation system comprising of a pedicle screw navigator, a first reference device, a second reference device and a surgical orientation device. The present invention provides methods using the pedicle screw navigation system to assist in determining the proper orientation of a pedicle screw during spinal surgery.

### BRIEF DESCRIPTION OF THE DRAWINGS

FIG. 1 shows a side view of a human vertebral column;

FIG. 2 shows a side view of a human vertebra;

FIG. 3 shows a top view of the human vertebra as shown in FIG. 2;

FIG. 4 shows a perspective view of an embodiment of a vertebral coupler of a spinal alignment system of the present invention;

FIG. 5 shows a side view of the vertebral coupler showed in FIG. 4 attached to the spinous process of the human vertebra shown in FIG. 2;

FIG. 6 shows a perspective view of another embodiment of a vertebral coupler of the spinal alignment system of the present invention;

FIG. 7 shows a perspective view of the vertebral coupler showed in FIG. 6 attached to the spinous process of the human vertebra shown in FIG. 2;

FIG. 8 shows a perspective view of the vertebral coupler attached to the spinous process of the human vertebra as shown in FIG. 5 next to a reference device of the spinal alignment system of the present invention;

FIG. 9 shows a back view of the reference device shown in FIG. 8;

FIG. 10 shows an exploded view of the reference device shown in FIG. 8;

FIG. 11 shows a perspective view of five of the reference devices shown in FIG. 8 each coupled to the vertebral coupler shown in FIG. 5;

FIG. 12 shows a perspective view of five of the reference devices shown in FIG. 8 each coupled to the vertebral coupler shown in FIG. 4 wherein each of the vertebral coupler is attached to the transverse process of the human vertebra shown in FIG. 3;

FIG. 13 shows a perspective view of a surgical orientation device of the spinal alignment system of the present invention;

FIG. 14 shows a front view of the surgical orientation device shown in FIG. 13;

FIG. 15 shows an exploded view of the surgical orientation device shown in FIG. 13;

FIG. 16 shows a perspective view of the human vertebra shown in FIG. 2;

FIG. 17 shows a perspective view of a pedicle screw navigator of the pedicle screw navigation system of the present invention; and

FIG. 18 shows a perspective view of the human vertebra as shown in FIG. 16 attached to the pedicle screw navigator as shown in FIG. 17.

### DETAILED DESCRIPTION OF THE PREFERRED EMBODIMENT

The present invention provides devices and methods to assist in alignment of the spine during a surgical procedure to correct a deformity due to trauma or degeneration. Standard medical practice is to fuse one or more levels of the spine by immobilization. This immobilization is accomplished by implanting plates, rods, or other devices that are rigidly fixed to two or more vertebrae.

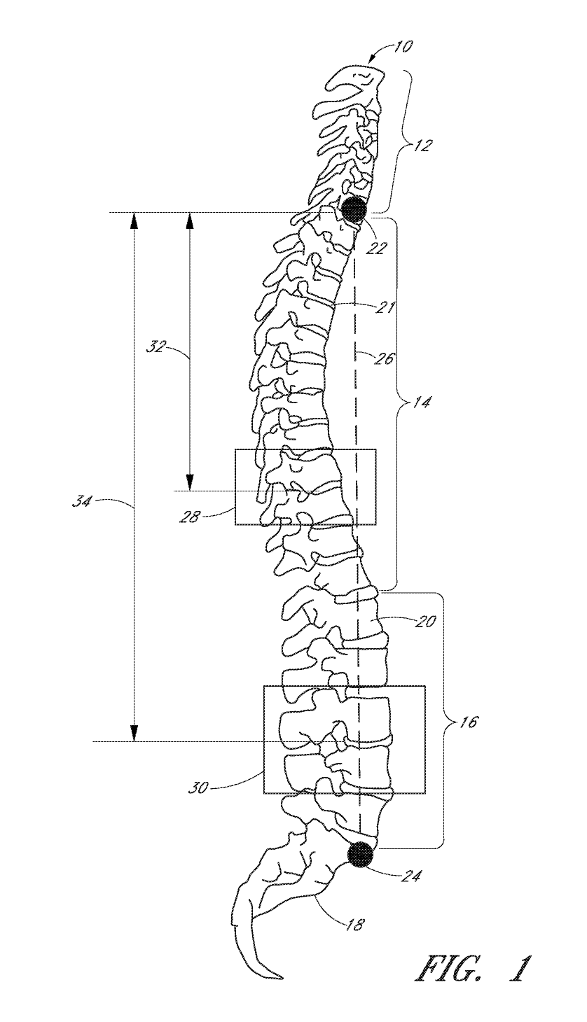

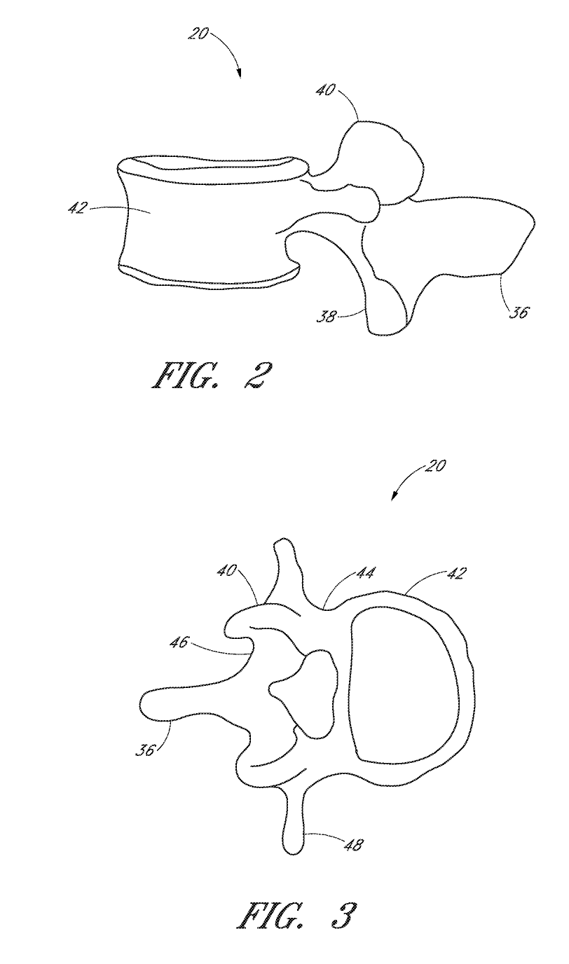

With reference to FIG. 1, standard practice is to fuse one or more levels 28 & 30 of the spine 10 by immobilization. This immobilization is accomplished by implanting plates, rods, or other devices that are rigidly fixed to two or more vertebrae 20. This procedure is most often performed in the thoracic 14 or lumbar 16 regions of the spine 10. The intervertebral disc(s) 21 within the fusion region 28 are removed to allow direct apposition of adjacent vertebral bodies 42, promoting bony growth. The spinous process 36, pedicles 44, and articular processes 38 & 40 are routinely used as fixation points.

Prior to operating, the surgeon consults imaging (plain radiographs, CT, or MR imaging) to determine the degree of deformity in the various anatomic planes. More specifically, the overall sagittal alignment of the affected spine 10 is evaluated via a sagittal axis 26 drawn between the reference points on the C7 vertebra 22, and on the sacrum 24. In the normal spine, this axis 26 is vertical. One goal of surgery, therefore, is to apply an angular correction to the spine in order to bring the sagittal axis 26 into vertical alignment. In the coronal plane, the ideal alignment can be defined as the centers of all vertebrae 20 lying on a line. Similarly, ideal axial alignment (rotation in the transverse plane) consists of all spinous processes 36 extending from a central axis at a common angle.

With these goals in mind, the surgeon determines locations for implant fixation, and the amount of angular correction desired between each instrumented level. Exemplary areas of adjustment 28 and 30 are shown in FIG. 1. For each area of adjustment (28, 30), the surgeon calculates or estimates the angular correction required to bring the spine into the desired alignment. Since there are infinitely many solutions to restoring the sagittal axis 26 to vertical alignment, areas of adjustment 28 & 30 are chosen based on anatomical considerations and the surgeon's experience. These correction angles are the targets used during surgery to determine when sufficient adjustment has been made. These angles are subsequently referred to herein as “correction targets”. Optionally and referring to FIG. 1, the surgeon may also measure and record the vertical distances 32 & 34 from the centers of the areas of adjustment to the cranial reference point 22 of the sagittal axis 26.

### I. The Spinal Alignment System

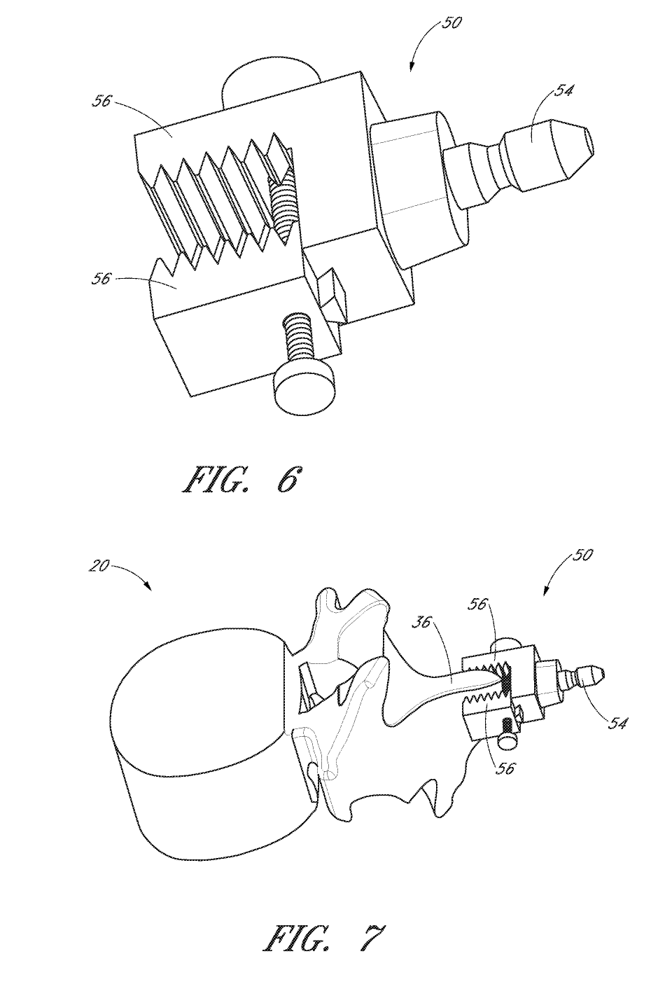

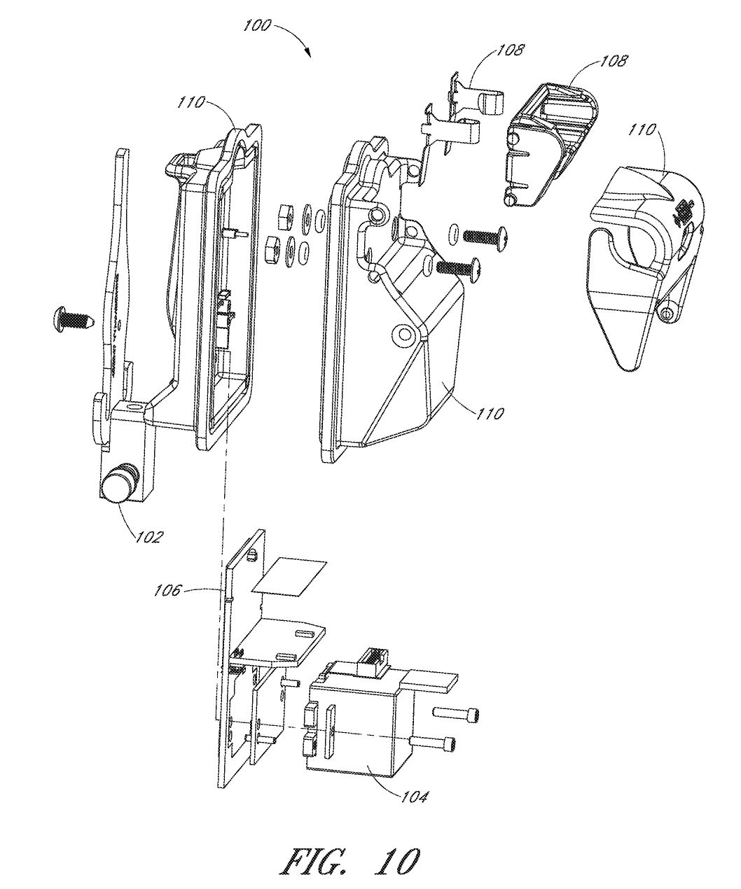

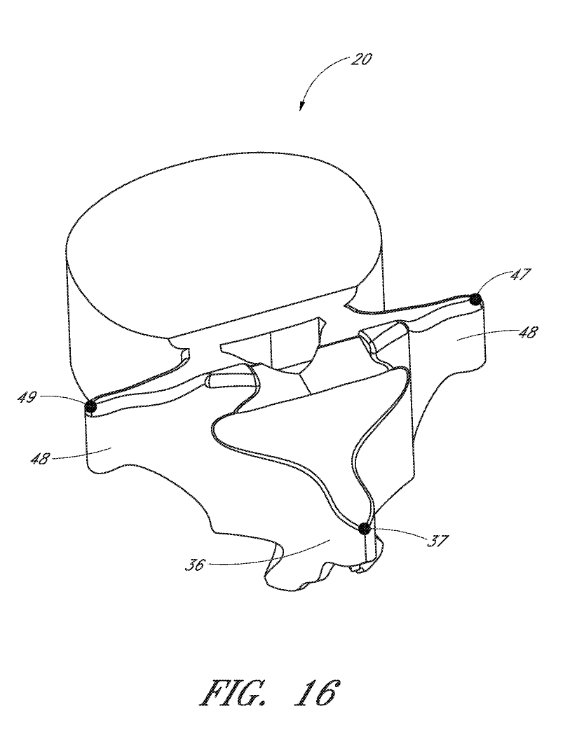

Referring to FIGS. 4-15, the present invention provides, in certain embodiments, a spinal alignment system comprising of at least one vertebral coupler 50, at least one reference device 100 and a surgical orientation device 200. The reference device 100 incorporates generally the same components and basic measurement functions as described in U.S. Pat. No. 8,118,815 for its reference device (e.g., 16) with one or more processors or other physical computer hardware that can implement software modifications as required to fulfill the basic software requirements described below. The surgical orientation device 200 incorporates generally the same components and basic measurement functions as described in U.S. Pat. No. 8,118,815 for its surgical orientation device (e.g., 14) with one or more processors or other physical computer hardware that can implement software modifications as required to fulfill the basic software requirements described below. U.S. Pat. No. 8,118,815 is hereby incorporated by reference in its entirety

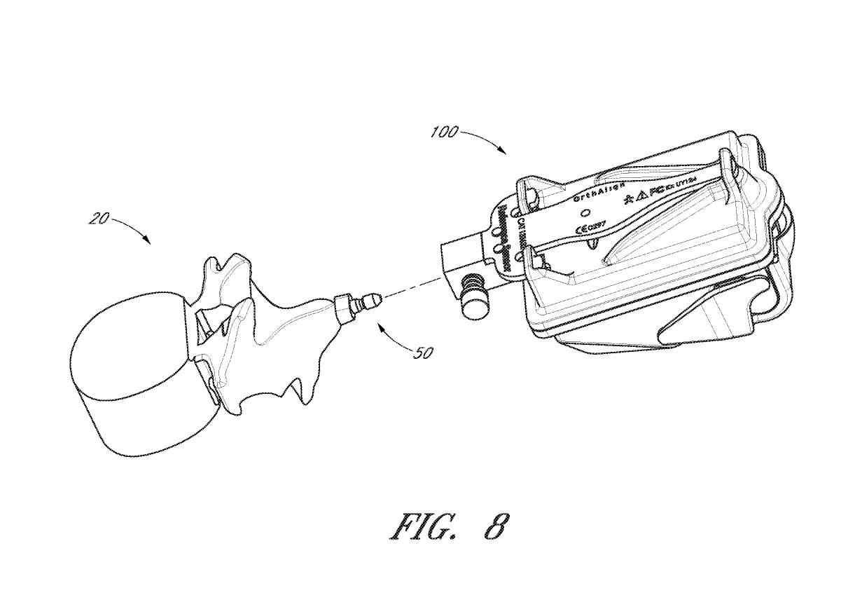

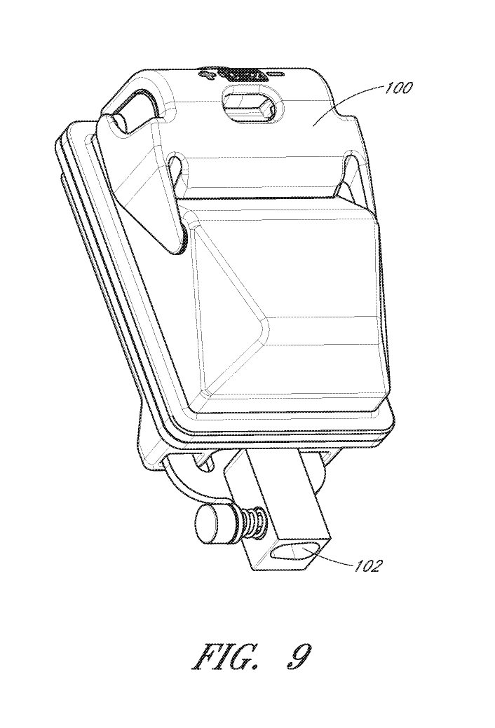

Referring to FIG. 4-7, the vertebral coupler 50 includes a reference coupler feature 54 capable of securely connecting to the corresponding vertebral coupler feature 102 of the reference device 100, constraining the position and orientation of the attached reference device 100. In one exemplary embodiment, the vertebral coupler 50 further includes a screw feature 52 of appropriate length, diameter, and thread form as shown in FIG. 4 to achieve secure fixation to the spinous process 36 (or any other appropriate site such as pedicle, transverse process, or the like) of the vertebra 20 as shown in FIG. 5. In another exemplary embodiment, the screw feature 52 is replaced by two or more bone clamping features 56 configured to oppose each other as shown in FIGS. 6-7.

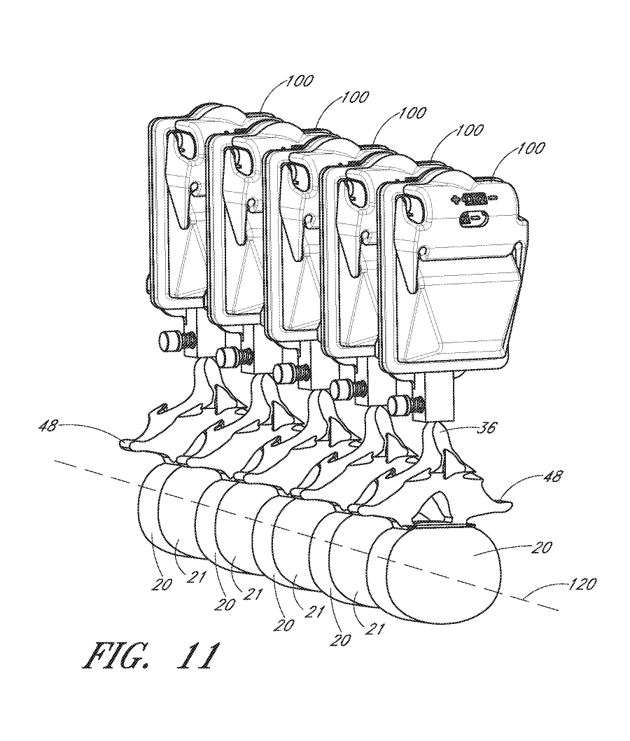

Referring to FIGS. 9-10, the reference device 100 includes in some embodiments, at minimum, the following major components, or similar components capable of performing generally the same functions: a vertebral coupler feature 102 configured to connect or couple to the vertebral coupler 50; at least one accelerometer capable of measuring its orientation relative to the acceleration due to gravity; in this case, an inertial measurement unit 104 containing multiple accelerometers; a circuit board 106 containing a digital signal processor to interpret the signals from the accelerometers and a radio to communicate with the surgical orientation device 200 and other reference devices (100); a power supply interface 108 adapted to connect to a power supply (e.g., battery) to supply power to the reference device 100; a housing 110 to enclose and protect the electronic components of the reference device 100; and one or more processors or other physical computer hardware that can implement software capable of performing the following functions: (i) gathering measurements from the accelerometers; (ii) performing calculations to convert accelerometer signals to angular orientation; and (iii) transmitting data to the surgical orientation device 200.

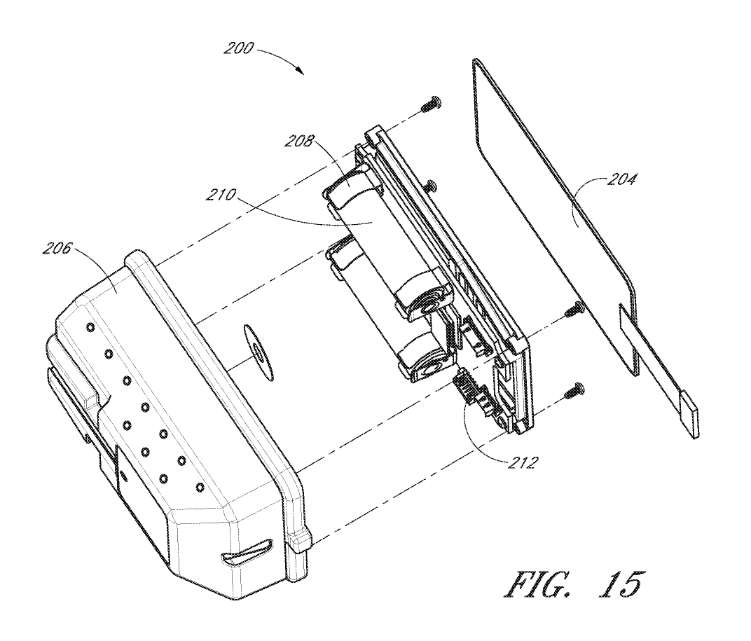

Referring to FIGS. 13-15, the surgical orientation device 200 includes in some embodiments, at minimum, the following major components, or similar components capable of performing generally the same functions: an LCD 202 to display alignment data to the user; a keypad (or push buttons) 204 to allow inputs to the surgical orientation device 200; a circuit board 212 containing a processor to drive the surgical orientation device 200 and calculate the relative angle of the reference device(s) 100 based on their individual inputs; a power supply interface 208 adapted to connect to a power supply (e.g., batteries 210) in order to supply power to the surgical orientation device 200; a housing 206 to enclose and protect the electronic components of the surgical orientation device 200; and one or more processors or other physical computer hardware that can implement software capable of performing the following functions: (i) establishing communications with and receiving data from the reference devices 100; (ii) receiving user inputs from the keypad 204; (iii) performing the necessary calculations (e.g., trigonometric calculations) such as converting angular measurements from one reference frame to another; (iv) retaining data such as calculation results and user inputs in memory; and (v) displaying data such as calculation results or reference images on the LCD 202. More generally, the software described herein can be implemented in physical computer hardware including, for example, one or more processors, a memory, physical computer storage, and the like.

In one alternative embodiment, the surgical orientation device 200 also includes the components (e.g., accelerometers) and one or more processors or other physical computer hardware that can implement software necessary for it to also function as the reference device 100.

### II. Methods of Spinal Alignment

During surgery, the patient is positioned prone on the operating table. After the typical exposure is made, the vertebral coupler(s) 50 are secured to two or more vertebrae 20 in a method as shown in FIG. 5 or 7. The vertebral couplers 50 can be placed on any desired vertebra 20. For example, it is not necessary that the vertebral couplers 50 are placed on consecutive vertebrae 20. The vertebrae 20 to which the couplers 50 are mounted are those previously selected for fixation, and between which correction targets have been established. The vertebrae 20 may be first prepared by drilling a pilot hole prior to securing the vertebral coupler 50. The hole for the vertebral coupler 50 is preferably made in the spinous process 36, entering either posteriorly as shown in FIG. 5 or laterally. Instead of attachment to the spinous process 36, the vertebral coupler 50 can be attached to the vertebral body 42, to the transverse processes 48, to a screw placed in the pedicle 44, or any other part of the vertebra 20 as desired by the user.

Instead of attaching the vertebral coupler 50 directly to the vertebra 20 by a threaded element 52 as shown in FIG. 5, a vertebral coupler 50 with clamping features 56 as shown in FIG. 6 may be fixed to the spinous process 36 as shown in FIG. 7, or to (i) the vertebral body 42, (ii) the transverse processes 48, (iii) a screw placed in the pedicle 44, or (iv) any other part of the vertebra 20 as desired by the user.

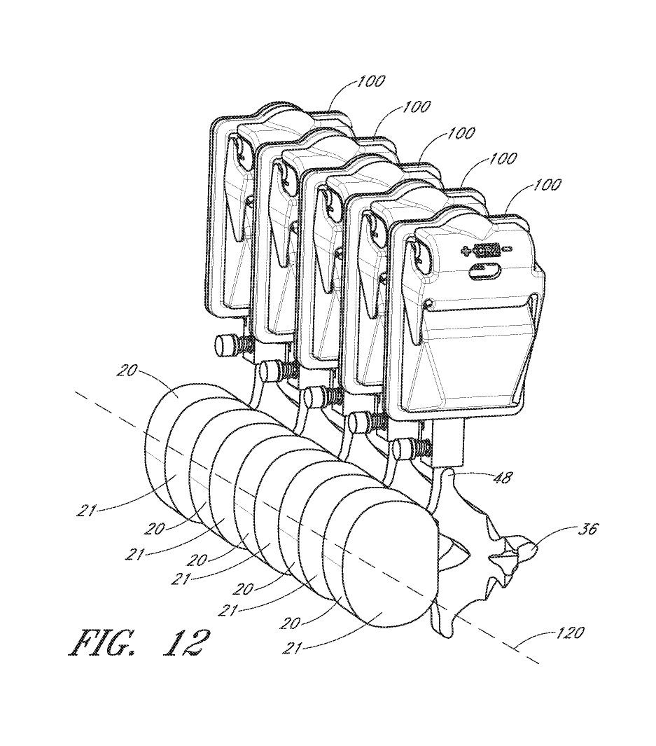

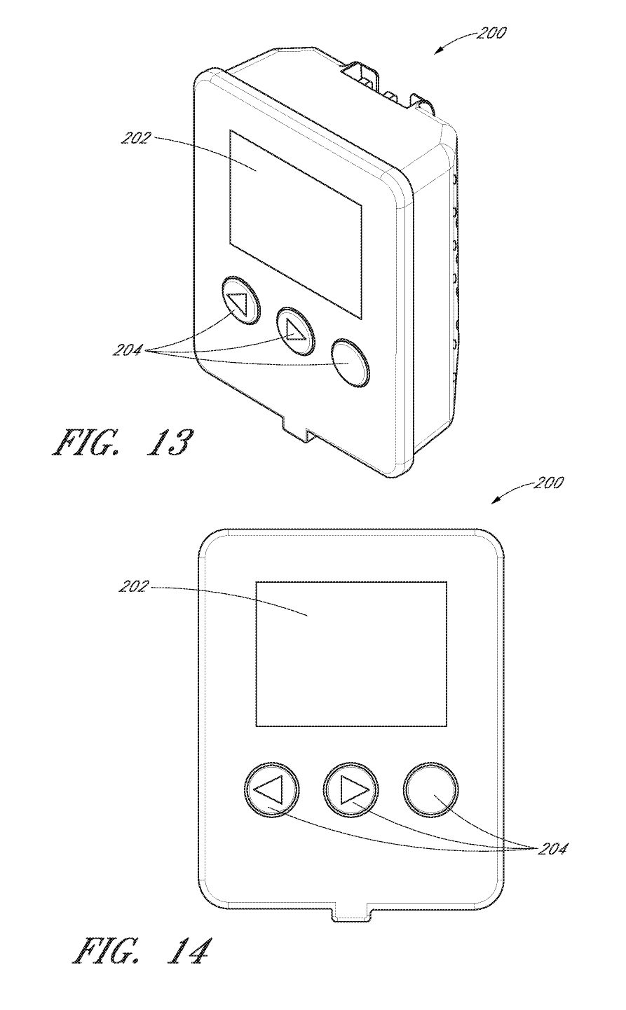

The vertebral couplers 50 may be attached to adjacent vertebrae 20 as shown in FIGS. 11-12, or non-adjacent vertebrae 20. If non-adjacent vertebrae 20 are instrumented, the cumulative angular change at all included levels within the area of adjustment 28 & 30 will be measured. Each pair of reference devices 100 defines an area of adjustment 28 & 30.

After the vertebral couplers 50 are securely fixed to the selected vertebrae 20, the reference device 100 is attached to each of the vertebral couplers 50 as shown in FIGS. 11-12. The vertebral couplers 50 constrain the rotation and position of the reference devices 100, which are, therefore, rigidly fixed to the vertebrae 20 such that a change in angle of each vertebra 20 relative to gravity necessarily induces identical angular change in the corresponding reference device 100. Thus, the angle of each vertebra 20 (relative to gravity) is detected by its reference device 100.

To achieve maximum measurement accuracy, all of the reference devices 100 should be oriented along the axis of the spine 120, as shown in FIGS. 11-12. This may be accomplished by a number of methods. For example, a laser mounted in one of the reference devices 100 (as implemented in the KneeAlign®™ device commercialized in 2010 by OrthAlign® located in Aliso Viejo, Calif.) creates a reference line for visually aligning the other reference devices 100. Alternatively, an elongated element (such as an orthopedic alignment rod) attached to one of the reference devices 100 would provide a similar reference. As yet another alternative, a radio beacon could be established at a reference point (e.g. last reference device 100 in the line, or a distant point on the spine). The signal strength received by directional antennae in the reference devices 100 would provide a quantitative alignment reference.

With all of the reference devices 100 in place on the spine 10, as shown in FIGS. 11-12, the user indicates to the surgical orientation device 200 (through inputs to the keypad 204) that the system should record the initial alignment. Each of the reference devices 100 then communicates its instantaneous orientation to the surgical orientation device 200 preferably through wireless transmission. Transmission may be accomplished via conventional signal transmission methodologies such as radio, visible light, infrared, or electrical transmission. The surgical orientation device 200 may be incorporated into one of the reference devices 100, or may be a separate device, as shown in FIGS. 13-15. The surgical orientation device 200 then uses the sagittal-plane angles of the reference devices 100 to calculate the relative angles between the vertebrae 20 of interest (those identified for correction pre-operatively). These angles are stored in the system memory of the surgical orientation device 200 as the pre-correction angles.

In one exemplary embodiment, the method of the present invention described above is used to track the sagittal plane adjustments of the spine 10. This method can also be used to track the transverse plane adjustment of the spine 10. Alternatively and in another exemplary embodiment, the method of the present invention allows the patient to be positioned laterally and the coronal plane and/or the transverse plane adjustments of the spine 10 are measured and displayed.

As the spine 10 is manipulated in the normal course of surgery (placement of fixation instrumentation), the surgical orientation device 200 continuously calculates the current sagittal-plane angle between the vertebrae 20 of interest. The difference between the current angle and the pre-correction angle for each of the reference devices 100 pair is the correction angle for that area of adjustment 28 & 30. The surgical orientation device 200 calculates and displays these correction angles throughout the course of surgery.

The surgeon follows normal practice to adjust the fixation instrumentation until the surgical orientation device 200 indicates that the desired corrections have been made. This occurs when the correction angles match the correction targets determined pre-operatively.

Referring to FIG. 1, the previously-measured vertical distances 32 & 34 from the areas of adjustment 28 & 30 to the cranial reference point 22 of the sagittal axis 26 may be input to the surgical orientation device 200 during surgery. With these distances, the surgical orientation device 200 may calculate the linear (anterior or posterior) shift in the cranial reference point 22 caused by the current (or a proposed) correction angle in a particular area of adjustment 28 & 30. For example, in area of adjustment 30, this anterior-posterior shift is equal to the product of distance 34 and the sine of the correction angle within area 30. These calculations may be used to intra-operatively adjust the correction targets as required, while still satisfying the ultimate goal of correct alignment of the axis 26.

With the fixation instrumentation in place, and adjusted to the desired correction, the reference devices 100 and vertebral couplers 50 are removed from the spine 10, and the surgery is completed following standard practice. The final alignment or correction angle for each area of adjustment 28 & 30 may be stored by the reference devices 100 and/or the surgical orientation device 200 prior to removing the reference devices 100 from the spine 10.

### III. Pedicle Screw Navigation System and Method

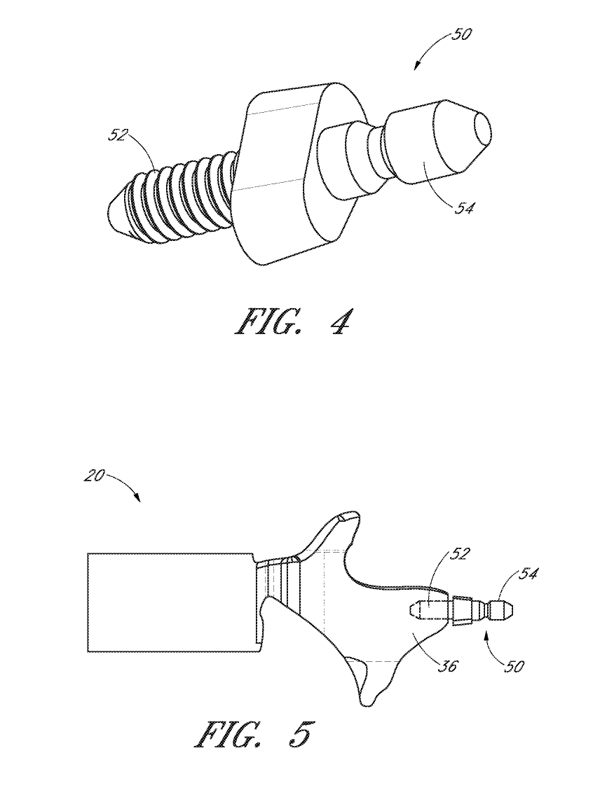

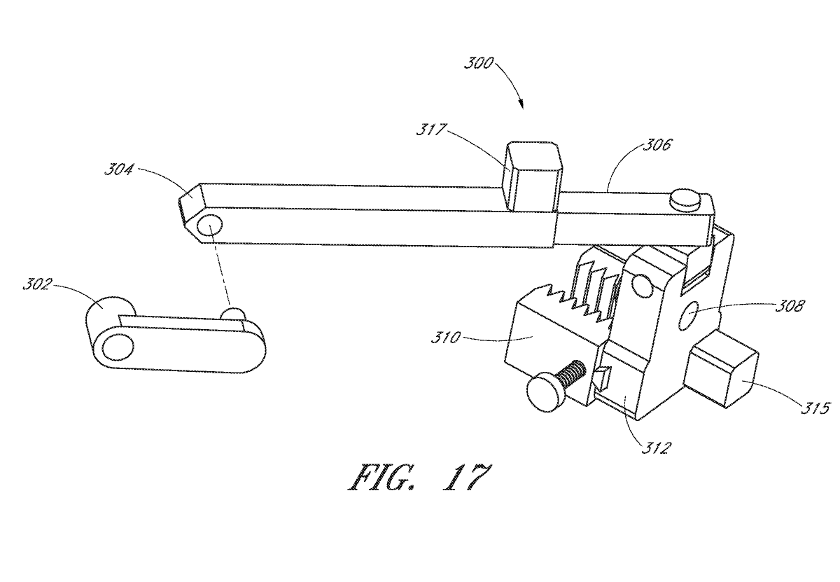

The present invention further provides a pedicle screw navigation system and methods for pedicle screw navigation during spinal surgery. Prior to operating, the surgeon consults imaging (plain radiographs, CT, or MR imaging) to determine ideal angle at which to place fixation screws (pedicle screws) into the pedicles 44 as shown in FIG. 3. Within the same imaging dataset, the surgeon also identifies three landmarks that are expected to be accessible within the surgical exposure. Referring to FIG. 16, these three landmarks 37, 47 and 49 may be, for example, the most cranial and lateral/posterior points on the transverse 48 and spinous process 36. The orientation of the ideal pedicle screw trajectory is recorded in terms of two angles (in the sagittal and transverse planes) from the plane defined by the three landmark points 37, 47 and 49.

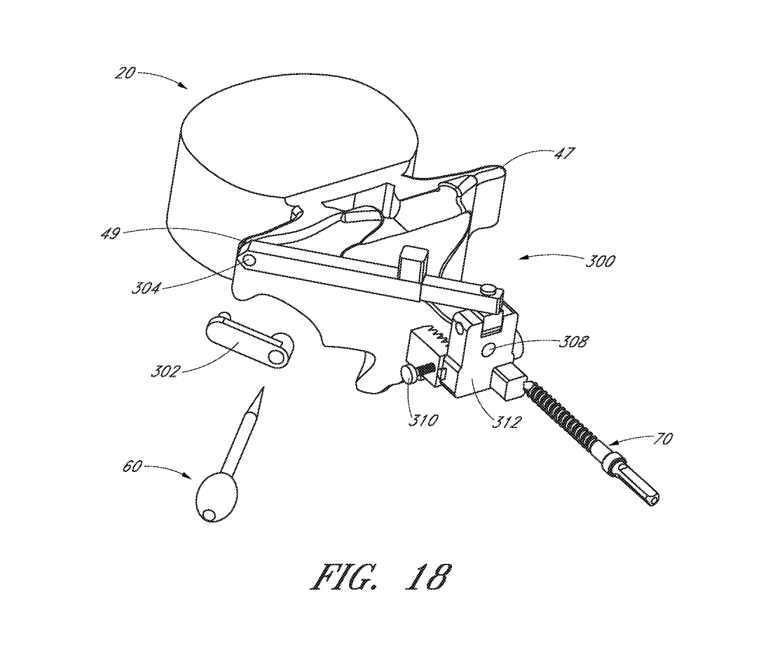

Following the normal course of surgery, the patient is positioned prone on the operating table, and an exposure is made using a posterior approach. The pedicle screw navigation system of the present invention comprising of a pedicle screw navigator 300, a first reference device 314, a second reference device 316 and a surgical orientation device 318. Referring to FIG. 17-18, the pedicle screw navigator 300 is attached to the vertebra 20 designated for pedicle screw mounting. The pedicle screw navigator 300 incorporates the following components and/or features: a fixed base 312, which can be rigidly fixed to the vertebra 20, and to which additional components can be mounted; a clamping element or elements 310, which facilitate fixation of the base 312 to the vertebra 20; a through hole 308 to accept a bone pin 70, which facilitates fixation of the base 312; a pivoting arm 306, which rotates relative to the base 312 in two directions approximately in the sagittal and transverse planes; a registration arm 304, which cannot rotate relative to pivoting arm 306, but can slide to change the effective length of the pivoting arm 306; a drill guide 302, which is mounted to the registration arm 304, and can rotate in a plane established by the registration arm 304. The drill guide 302 incorporates a through hole with an axis parallel to the drill guide's 302 rotation axis, which can accept a drill, an awl 60, or other instrument to create an opening or hole in the vertebra 20.

The pedicle screw navigation system includes the first reference device 314 (same as the reference unit 100 described above) which during use is rigidly fixed to the base 312 at first attachment feature 315 in a known orientation relative to the fixed base 312.

During use, this first reference unit 314 communicates its orientation to the surgical orientation device 318 (same as the surgical orientation device 200 described above). The pedicle screw navigation system includes the second reference device 316 (same as the first reference unit 314) which during use is rigidly fixed to the registration arm 304 at attachment feature 317 in a known orientation relative to the registration arm 304, and which communicates its orientation to the surgical orientation device 318.

Referring to FIG. 18, the pedicle screw navigator 300 is attached to the vertebra 20. The base 312 is positioned via a bone pin 70 passing through hole 308, and entering the spinous process 36 at landmark point 37. With the pin 70 secure, the clamp 310 is tightened to ensure rigid fixation to the vertebra 20. The fixed location of through hole 308 relative to the reference device 314 on base 312 provides the bearing of the first landmark point 37 relative to the reference device 314. The distal end of registration arm 304 is then serially rotated and extended into position at landmark points 47 and 49, with the user pausing at each position to enter these positions to the surgical orientation device 318. Upon input from the user, the surgical orientation device 318 stores the orientations of the reference device 316 mounted to the registration arm 304 relative to the reference device 314 mounted to the base 312.

With the known headings of the three landmark points 37, 47, and 49 from the reference device 314 on the base 312, the surgical orientation device 318 calculates the orientation of the base 312 relative to the vertebra 20 with its target pedicle screw trajectory identified pre-operatively. The surgical orientation device 318 next transforms that trajectory into the coordinate system of the reference device 314 mounted on the navigator base 312. From this point, the angles of the registration arm 304 from the target trajectory are continuously calculated and displayed to the user by the surgical orientation device 318.

The user next manipulates the registration arm 304 until the surgical orientation device 318 indicates that the registration arm 304 is correctly aligned with the target trajectory. Drill guide 302 is then attached to registration arm 304 and rotated (within the plane established by the registration arm 304) into position at the desired entry point of the pedicle screw (as visually determined by the surgeon). A drill or the awl 60 is introduced through the hole in drill guide 302, and used to create a hole into the vertebra 20.

Many other variations than those described herein and/or incorporated by reference will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm steps described in connection with the embodiments disclosed herein or incorporated herein by reference can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described or incorporated functionality can be implemented in varying ways for each particular application, but such implementation decisions should not be interpreted as causing a departure from the scope of the disclosure.

The various illustrative logical blocks and modules described in connection with the embodiments disclosed herein or incorporated by reference can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. A computing environment can include any type of computer system, including, but not limited to, a computer system based on a microprocessor, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others.

1. A pedicle screw placement system comprising:
a pedicle screw navigator having a first portion configured to temporarily mount to the patient and a second portion moveable relative to the first portion, the second portion configured to contact a posterior portion of vertebral anatomy;
a first reference device coupled with the first portion of the navigator;
a second reference device coupled with the second portion of the navigator; and
a user interface;
wherein the pedicle screw navigation system is configured to guide the insertion of a pedicle screw based at least in part on acquisition of anatomical landmarks using the pedicle screw navigator.

2. The pedicle screw navigation system of claim 1, wherein the first portion of the navigator comprises a mounting block to couple with a proximal portion of the vertebra and the second portion comprises a registration member moveably coupled with the mounting block.

3. The pedicle screw navigation system of claim 2, wherein the registration member is configured to pivot about a first axis and to be extended or retracted along a second axis.

4. The pedicle screw navigation system of claim 2, wherein the registration member is configured to contact at least two lateral anatomical landmarks of a vertebra.

5. The pedicle screw navigation system of claim 2, wherein the registration member is configured to contact first and second transverse processes of a vertebra.

6. The pedicle screw navigation system of claim 1, wherein the first portion of the pedicle screw navigator is adapted to apply a compressive force on a first portion of a vertebra.

7. The pedicle screw navigation system of claim 1, wherein the first portion of the pedicle screw navigator comprises a through hole configured to receive a pin to position the first portion along a first axis.

8. The pedicle screw navigation system of claim 7, wherein the first portion of the pedicle screw navigator comprises a jaw member to temporarily hold the first portion on a vertebra.

9. The pedicle screw navigation system of claim 1, wherein the user interface is housed in a hand-held device having circuitry configured to interact with the first and second reference devices.

10. The pedicle screw navigation system of claim 9, wherein the hand-held device comprises an inertial measurement unit.

11. An intraoperative orthopedic alignment system comprising:
at least one vertebral coupler;
at least one reference device; and
a surgical orientation device;
wherein the surgical orientation device is configured to display orientation of the at least one reference device during a procedure as an indication of alignment of one or more vertebrae of the spine.

12. The system of claim 11, wherein the reference device comprises at least one accelerometer adapted to measure acceleration relative to acceleration due to gravity.

13. The system of claim 11, further comprising:
an inertial measurement unit containing multiple accelerometers;
a digital signal processor to interpret the signals from the accelerometers;
a radio to communicate with the surgical orientation device;
a power supply interface adapted to connect to a power supply to supply power to the reference device;
a housing to enclose and protect the electronic components of the reference device; and
a processor configured to perform one or more of the following functions:
(i) gathering measurements from the accelerometers;
(ii) performing calculations to convert the measurements from the accelerometers to angular orientation; and
(iii) transmitting data representing the angular orientation to the surgical orientation device.

14. The system of claim 13, further comprising multiple reference devices, the radio adapted to communicate with the other reference devices.

15. The system of claim 13, wherein the surgical orientation device is adapted to communicate with multiple reference devices.

16. The system of claim 11, wherein the coupler comprises a first mounting feature on a first end and a second mounting feature on a second end, opposite the first end.

17. The system of claim 16, wherein the first mounting feature comprises a threaded length.

18. The system of claim 16, wherein the first mounting feature is configured to frictionally engage a proximal portion of a vertebra.

19. The system of claim 18, wherein the first mounting feature comprises a jaw device configured to apply a gripping force on lateral sides of a bony prominence of a vertebra.

20. The system of claim 16, wherein the reference device comprises a third mounting feature, the second and third mounting features configured to detachably couple to enable the reference device to be mounted on the coupler.
